(19) United States
(12) Patent Application Publication
(10) Pub. No.: US 2012/0237503 A1
(43) Pub. Date: Sep. 20, 2012

(54) Screening Method and Therapy with Agonists of DDAH I

(75) Inventors: Rajeshwar P. Mookerjee, London (GB); Rajiv Jalan, London (GB); Gautam Mehta, London (GB)

(73) Assignee: UCL BUSINESS PLC, London (GB)

(21) Appl. No.: 13/394,876

(22) PCT Filed: Sep. 9, 2010
(86) PCT No.: PCT/GB10/01704

§ 371 Date: Jun. 6, 2012

(30) Foreign Application Priority Data
Sep. 9, 2009 (GB) 0915794.2

(51) Int. Cl. A61K 39/395; A61K 31/7088; C12Q 1/68; A61P 7/00; C12Q 1/34; G01N 33/573

(52) U.S. Cl. 424/133.1; 435/18; 435/7.92; 435/6.12; 514/44 R

(57) ABSTRACT

The present invention derives from the finding that decreased levels of DDAH I are associated with increased portal pressure and that by increasing DDAH I levels in vivo, portal pressure may be reduced. Accordingly, the invention provides methods for reducing portal blood pressure comprising administering to a subject in need thereof an agonist of DDAH I.

### FIELD OF THE INVENTION

The present invention derives from the unexpected finding that decreased levels of DDAH I are associated with increased portal pressure and that by increasing DDAH I levels in vivo, portal pressure may be reduced. The present invention utilises this finding to identify and provide DDAH I agonists that may be used to reduce portal pressure, for example in the treatment of portal hypertension.

### BACKGROUND TO THE INVENTION

Statistics from the NIH for the period 1976-80 suggest that deaths from liver cirrhosis in the US were greater than 26,000. If this data is extrapolated to incorporate the increasing burden of viral and alcoholic liver disease currently in The West and also in the under-developed world, the number exceeds millions of cases per year world-wide. This figure is likely to continue to increase with the recognition of the new entity of Non-alcoholic fatty liver disease (in association with diabetes and the metabolic syndrome) which is increasingly recognized as a chronic liver disease with risk of progression.

Cirrhosis is associated with severe morbidity and mortality largely from portal hypertension. Increased blood pressure in the portal blood vessels may result from either increased volume of blood flowing through the vessels or/and increased resistance to the blood flow through the liver. In Western countries, the most common cause of portal hypertension is increased resistance to blood flow caused by extensive scarring of the liver in cirrhosis, which is most often due to chronic excessive alcohol intake.

Portal hypertension leads to the development of new veins (called collateral vessels) that directly connect the portal blood vessels to the general circulation, bypassing the liver.

Because of this bypass, substances (such as toxins) that are normally removed from the blood by the liver can pass into the general circulation. Collateral vessels develop in particular at the lower end of the esophagus and at the upper part of the stomach. Here, the vessels can become variceal. These engorged variceal vessels are fragile and prone to bleeding, sometimes seriously and occasionally with fatal results.

Current treatment to lower portal pressure to decrease risk from variceal bleeding is limited to about 40% efficacy, in part due to tolerability of agents such as beta-blockers. Moreover, there is a suggestion that such agents decrease liver perfusion which may further compromise liver function, as hepatic blood flow is already low in cirrhosis, despite systemic vasodilatation.

### SUMMARY OF THE INVENTION

The present invention relates to methods for the reduction of portal blood pressure.

In accordance with the present invention, this is achieved by administering an agonist of DDAH I, i.e. an agent that is capable of increasing or maintaining the activity or amount of DDAH I, particularly the activity or amount of DDAH I that is present in the liver of the subject to be treated.

Accordingly, the present invention provides a method of reducing portal blood pressure comprising administering to a subject in need thereof an agonist of DDAH I, wherein said agonist preferably does not reduce plasma TNFα levels in the subject.

The agonist may lead to (a) increased expression of DDAH I in the liver of the subject; and/or (b) increased levels of DDAH I in the liver of the subject. For example, the agonist may promote transcription of DDAH I in cells of the subject, or the agonist may be a vector capable of expressing DDAH I in the liver of the subject.

The agonist may increase the activity of DDAH I in the liver of the individual. Thus, the agonist may lead to (a) decreased levels of asymmetric dimethylarginine (ADMA); and/or (b) increased levels of nitric oxide synthase (NOS); in the liver of the individual.

The subject to be treated may be any subject in need of reduced portal pressure, such as a subject having portal hypertension. The subject may have liver cirrhosis.

The present invention also provides screening methods that can be used to identify suitable DDAH I agonists for use in these methods of treatment. Accordingly, the invention provides a method for identifying an agent suitable for use in treating portal hypertension, the method comprising determining whether a test agent is capable of increasing or maintaining the amount or activity of DDAH I, wherein the ability to increase or maintain the amount or activity of DDAH I indicates that the compound may be suitable for use in treating portal hypertension.

The screening method may comprise a step of contacting a cell or tissue comprising DDAH I with a test agent and determining whether the presence of the test agent leads to an increase in the amount or activity of DDAH I in the cell or tissue.

The amount or activity of DDAH I may be assessed in the liver or in tissue or cells derived from the liver. For example, the screening method may be carried out in a bile duct ligated rat and the method may comprise the steps of administering a test agent to a bile duct ligated rat and determining whether the presence of the test agent leads to an increase in the amount or activity of DDAH I in the liver of the rat.

A screening method of the invention may further comprise determining whether the test agent is capable of inhibiting TNFα0, wherein the ability to increase the amount or activity of DDAH I in combination with the absence of an inhibitory effect on TNFα indicates that the compound may be suitable for use in treating portal hypertension.

### BRIEF DESCRIPTION OF THE DRAWINGS

FIG. 1 reports on the 14C-citrulline counts per minute per milligram of protein. This is a measure of eNOS activity. Experiments were carried out in bile-duct ligated (BDL) or sham treated rats. The bile duct ligated rats were treated with either infliximab (BDL+infliximab) or a vehicle (BDL). This Figure shows that bile duct ligation markedly reduces eNOS activity, but that subsequent treatment with infliximab restores eNOS activity towards similar levels to those seen in the sham animals.

FIG. 2 reports ADMA levels in liver tissue in sham or BDL rats. The BDL rats were treated either with infliximab (BDL+INF) or with vehicle (BDL). Bile duct ligation led to a significantly elevated tissue ADMA concentration compared with sham animals. However ADMA levels were substantially reduced by treatment with infliximab. It is of interest that the relative concentration/accumulation of ADMA in the hepatic tissue in BDL animals is far greater than plasma ADMA concentration differences compared to sham animals.

FIG. 3 illustrates the markedly reduced expression of the DDAH-I isoform in livers of BDL rats. Upon treatment with Infliximab, the DDAH-I expression levels were restored towards sham levels.

FIG. 4 reports portal pressure in sham or BDL rats. Portal pressure was markedly increased in BDL cirrhotic rats compared to normal sham portal pressures (14±0.7 vs. 5.5±0.3 mmHg). Following intervention with Infliximab, this was reduced by more than 30% (9.5±0.6 mmHg).

FIGS. 5 and 6 report on hepatic eNOS activity (FIG. 5) and hepatic eNOS protein expression (FIG. 6). It was found that eNOS activity was significantly decreased in BDL animals compared to sham (*-p<0.05) despite increased eNOS protein expression (**-p<0.01). Following treatment with INT-747, eNOS activity reverted to sham levels (*-p<0.05), with similar normalisation of eNOS protein expression (*-p<0.05).

FIGS. 7 and 8 report on hepatic ADMA protein expression (FIG. 7) and hepatic DDAH1 protein expression (FIG. 8). It was found that ADMA expression was significantly increased in BDL animals (**-p<0.01), concomitant with significantly reduced DDAH-I protein expression (**-p<0.01). Following INT-747 administration DDAH-I expression increased significantly (**-p<0.01) with a significant reduction in ADMA (*-p<0.05) compared with BDL alone.

FIG. 9 reports portal pressure in sham or BDL rats. Portal pressure was significantly increased in BDL rats compared to sham (***-p<0.0001). Following INT-747 treatment there was a 30% reduction in portal pressure when compared to BDL+vehicle (**-p<0.01).

FIG. 10 reports the effects of reconstitution of DDAH I cDNA on portal pressure in sham or BDL rats.

### DETAILED DESCRIPTION OF THE INVENTION

Dimethylarginine dimethylaminohydrolase (DDAH) is an enzyme found in all mammalian cells. Two isoforms exist, DDAH I and DDAH II, with some differences in tissue distribution of the two isoforms. DDAH degrades methylarginines, specifically asymmetric dimethylarginine (ADMA) and NG-monomethyl-L-arginine (MMA). The methylarginines ADMA and MMA inhibit the production of nitric oxide synthase. Accordingly, DDAH is important in removing methylarginines, generated by protein degradation, from accumulating and inhibiting the generation of nitric oxide.

DDAH II has previously been associated with the degree of expression of endothelial NO synthase and also with angiogenesis. The DDAH isoform DDAH II has therefore been a target of previous studies. The inventors have now unexpectedly found that expression of the DDAH I isoform is significantly reduced in established models of cirrhosis (such as bile duct ligated rats—BDL, compared to sham rats). Expression of the DDAH I isoform is found to be further decreased in the context of superadded inflammation/infection (through endotoxin challenge). Moreover, the inventors have found that agonism of DDAH I, either by increasing its activity or by increasing its expression, leads to an increase in eNOS activity, a decrease in levels of methylarginines (such as ADMA) and a significant lowering in portal pressure.

The present invention therefore lies in the augmentation of DDAH I expression and/or activity in order to lower hepatic ADMA, increase liver NO generation and lower portal pressure. This has utility in the treatment of increased portal hypertension in a clinical setting, particularly in cirrhosis where increased portal hypertension is directly linked to increased variceal bleeding.

### DDAH I Agonists

The present invention relates to the agonism of DDAH I. An agonist of DDAH I may be any compound or molecule that increases the activity, function or amount of DDAH I. Preferably the agonist has its function in the liver of the patient. Preferably the agonist leads to an increase in DDAH I activity, function or amount in the liver of an individual to whom the agonist is administered. The agonist may act preferentially in the liver or may act at a number of locations including the liver. The agonist may be targeted to the liver during administration as discussed further below.

Preferred agonists are those that increase the activity or amount of DDAH I by at least 10%, at least 20%, at least 30%, at least 40%, at least 50%, at least 60%, at least 70%, at least 80% or at least 90% compared to the amount seen in the absence of the agonist. For example, increases of these sizes may be seen in the liver or liver tissue of a subject to whom the agonist has been administered.

The agonist may act specifically to agonise DDAH I. That is, the effect of the agonist on DDAH I may be greater than any other biological effect of the agonist. Such an agonist may be specific to the activation of DDAH I, that is it may increase or maintain the activity of DDAH I, but not other enzymes. Such an agonist may be specific to the expression of DDAH I, that is it may increase or maintain the expression of DDAH I but not other proteins. An agonist for use in accordance with the present invention may be an agonist of DDAH I as described herein, that does not act as an antagonist of TNFα. An agonist for use in accordance with the present invention may act on DDAH I in preference to DDAH II. For example, an agonist of DDAH I for use in accordance with the present invention may have one or more of the characteristics of a DDAH I agonist as described herein, but may not have such characteristics in relation to DDAH II, or may have such characteristics to a lower level in relation to DDAH II when compared to DDAH I. For example, an agonist that increases the activity of DDAH I may not increase the activity of DDAH II, or may increase the activity of DDAH II to a lesser extent, such as a lower percentage increase, than its effect on DDAH I. An agonist that increases the expression or amount of DDAH I may not increase the expression or amount of DDAH II, or may increase the expression of DDAH II to a lesser extent, such as a lower percentage increase, than its effect on DDAH I

Any agent capable of stimulating the activity or function of DDAH I may be suitable for use in the methods of the present invention. The primary function of DDAH I is the enzymatic degradation of methylarginines such as asymmetric dimethylarginine (ADMA). An agonist of the present invention may therefore act to increase the degradation of methylarginines by DDAH I, such as the degradation of ADMA. An agonist of the invention may act to increase the activity, function or amount of DDAH I and thereby lead to a decrease in the level of methylarginines such as ADMA, preferably in the liver. Agonist activity may therefore be identified by the ability to cause a decrease in ADMA levels in the liver.

Methylarginines such as ADMA inhibit the production of nitric oxide synthase (NOS). Accordingly, an agonist of the present invention may act to increase the activity, function or amount of DDAH I and thereby increase levels of NOS, preferably in the liver. Agonist activity may therefore be identified by the ability to increase levels of NOS in the liver.

Agonists for use in accordance with the present invention may be direct or indirect agonists of DDAH I.

Direct agonists are agents whose activity is directly on DDAH I. For example, direct agonists may be agents that act directly on the DDAH I enzyme to increase or to maintain its activity. A direct agonist may be an agent that prevents degradation of DDAH I or increases its half life in vivo. A direct agonist may be an agent that stabilises the DDAH I enzyme. A direct agonist may increase the amount of DDAH I by providing additional DDAH I to the patient. A direct agonist may be an agent that acts on the DDAH I gene, promoter or other gene regulatory regions to increase or maintain expression of DDAH I. A direct agonist may increase or maintain expression of DDAH I by stimulating or maintaining expression from the endogenous DDAH I gene. A direct agonist may increase or maintain expression of DDAH I by providing additional DDAH I gene(s) to the relevant cell so that expression may be achieved from such additional polynucleotides.

For example, the Farnesoid receptor agonist INT-747 is a bile-acid responsive nuclear receptor which promotes the expression of several target genes including DDAH I. As shown in the Examples, the inventors have shown that INT-747 acts to increase DDAH I expression in the liver. This increase in DDAH I levels leads to a concomitant decrease in ADMA levels in the liver tissue, an increase in NOS activity and ultimately a decrease in portal pressure. An agonist of DDAH I for use in accordance with the present invention may therefore be an agent that promotes the expression of DDAH I, such as a Farnesoid receptor agonist, such as the Farnesoid receptor agonist INT-747.

Indirect agents are agents whose activity leads to agonism of DDAH I, but which do not act directly on DDAH I. For example, indirect agonists include agents such as anti-inflammatory agents or anti-TNFα agents that may stimulate DDAH I as a downstream effect. An agonist of DDAH I may therefore be an agent that has effects which lead to an increase in DDAH I activity, function or amounts.

For example, the inventors have found that infliximab, an anti-TNFα antibody, is capable of increasing DDAH I levels in the livers of bile duct ligated rats. As shown in the Examples, this increase in DDAH I levels leads to a concomitant decrease in ADMA levels in the liver tissue, an increase in NOS activity and ultimately a decrease in portal pressure.

Any of the agonists described herein may therefore be used to agonise DDAH I, i.e. to increase the amount of DDAH I that is present, and/or the activity or the function of the DDAH I. Preferably these agonising effects take place in the liver.

Some agents that are capable of agonising DDAH I may be unsuitable for in vivo administration as part of a treatment as described herein. Some agents that are capable of agonising DDAH I, particularly some indirect agonists may have other unwanted effects on the patient. A physician will be able to balance for an individual patient whether those unwanted side-effects outweigh the potential benefits of the DDAH I agonism described herein in order to select a suitable DDAH I agonist for use as described herein.

For example, Naveau et al (Hepatology (2004) 39: 1390-1397) reported that systemic administration of infliximab in association with prednisolone to patients with acute alcoholic hepatitis led to a high level of severe infections which was considered unacceptable. The agonist for use in accordance with the present invention may therefore be an agent that agonises DDAH I as described herein, but that does not have an immunomodulatory effect. For example, the agonist may be an agent that agonises DDAH I as described herein, but that does not reduce levels of pro-inflammatory cytokines and/or that does not have an anti-TNFα effect. The agonist may have no effect on the levels of pro-inflammatory cytokines in vivo and/or may have no effect on the level of plasma TNFα in vivo. For example, the agonist may be an agent that agonises DDAH I as described herein which is not an anti-TNFα antibody such as infliximab.

An agonist of DDAH I may be an agent that increases the production of endogenous DDAH I. For example, the agent may act within the cells of the subject to enhance or stimulate the expression of DDAH I. Such an agent may be a transcription factor or enhancer that acts on the DDAH I gene to promote gene expression. For example, such an agent may be a nuclear receptor such as the Farnesoid receptor agonist INT-747.

An agonist of DDAH I may be an agent that provides the cells of an individual with the ability to produce additional DDAH I. For example, the agent may be a vector that is capable of expressing DDAH I such as an expression vector comprising a DDAH 1 gene and other sequences as necessary for expression of that gene. Thus, DDAH I may be provided by delivering such a vector to a cell and allowing transcription from the vector to occur. The agent may be a polynucleotide that is capable of expressing DDAH I such as a vector comprising a DDAH I gene and other sequences as necessary for integration of the DDAH I gene into the host genome and to allow expression from that inserted DDAH I gene sequence. Methods for gene delivery are known in the art. See, e.g., U.S. Pat. Nos. 5,399,346, 5,580,859 and 5,589,466. The polynucleotide may be expressed under the control of a suitable promoter. For example, expression of the DDAH I polynucleotide may be targeted to the liver by using a liver specific promoter. Thus, the agonist may be a polynucleotide or vector comprising a DDAH I gene and a liver specific promoter. For example, as described in Example 4, the liver specific LPI promoter may be used. As described in Example 4, hepatic restricted transgene production may be achieved by using a truncated liver specific promoter, LPI, containing segments of the human apoE/CI hepatic control region (HCR) and alpha-1 -antitrypsin (hAAT) gene promoter, as described in Osman et al. Atherosclerosis 2009, 204: 121-6.

The nucleic acid molecule can be introduced directly into the recipient subject, or can be introduced ex vivo into cells which have been removed from a subject. In this latter case, cells containing the DDAH I vector may be re-introduced into the subject at a suitable location, such as to the liver of the subject. Various approaches for such gene delivery are known in the art and would be appreciated by the skilled reader. For example, a DDAH I gene could be delivered as a naked nucleic acid construct, preferably further comprising flanking sequences homologous to the host cell genome. The DDAH I gene could be delivered in a vector such as a plasmid vector, or a viral vector. Suitable recombinant viral vectors include but are not limited to adenovirus vectors and adeno-associated viral (AAV) vectors. For example, transduction of hepatocytes and other cell types in rodent models of liver disease has been reported using adenovirus vectors (Yu et al. Am J Phys 2002, 282: G565-G572, Garcia-Banuelos et al. Gene Therapy 2002, 9: 127-134). Such adenovirus vectors may be used in accordance with the present invention. Similarly, liver transduction of the AAV2 genome with an AAV8 capsid (AAV2/8) has also been reported (Osman et al. Atherosclerosis 2009, 204: 121-6). Such AAV2/8 vectors may also be used in accordance with the present invention. The DDAH I gene could be administered in a liposomal preparation such as a cationic liposomal preparation.

### Screening Methods

The present invention also provides methods for the identification of agents suitable for use in the treatment of portal hypertension. For example, the invention provides methods for the identification of agonists of DDAH I which are suitable for use in lowering portal pressure. Agonists identified by this method may be agonists of DDAH I having any of the characteristics or effects described above.

Accordingly, the invention provides a method of identifying an agent for use in the treatment of portal hypertension, the method comprising determining whether a test agent is capable of increasing or maintaining the activity or expression of DDAH I. For example, the method may involve determining whether a test agent is capable of increasing or maintaining the amount or activity of DDAH I, wherein the ability to increase the amount or activity of DDAH I or the ability to maintain the amount or activity of DDAH I indicates that the compound may be suitable for use in treating portal hypertension.

A test agent for use in a screening method of the invention refers to any compound, molecule or agent that may potentially agonise DDAH I. The test agent may be, or may comprise, for example, a peptide, polypeptide, protein, polynucleotide, small molecule or other compound that may be designed through rational drug design starting from known agonists of DDAH I.

The test agent may be any agent having one or more characteristics of an agonist of DDAH I as described above. For example, a test agent may be an agent that is capable of increasing NOS levels in the liver.

The test agent to be screened could be derived or synthesised from chemical compositions or man-made compounds. Candidate agents may be obtained from a wide variety of sources including libraries of synthetic or natural compounds. Suitable test agents which can be tested in the above assays include compounds derived from combinatorial libraries, small molecule libraries and natural product libraries, such as display (e.g. phage display) libraries. Multiple test agents may be screened using a method of the invention in order to identify one or more agents having a suitable effect on DDAH I, such as stimulation of DDAH I activity or expression.

The screening methods of the invention may be carried out in vivo, ex vivo or in vitro. In particular, the step of contacting a test agent with DDAH I or with a cell or tissue that comprises DDAH I may be carried out in vivo, ex vivo or in vitro. The screening methods of the invention may be carried out in a cell-based or a cell-free system. For example, the screening method of the invention may comprise a step of contacting a cell or tissue comprising DDAH I with a test agent and determining whether the presence of the test agent leads to an increase in the amount or activity of DDAH I in the cell or tissue.

For example, the ability of a test agent to increase or maintain the activity or expression of DDAH I may be tested in a host cell or tissue that expresses DDAH I. For example, the amount or activity of DDAH I may be assessed in vitro, in vivo or ex vivo in the liver or in tissue or cells derived from the liver.

In such a cell-based assay, the DDAH I and/or the test agent may be endogenous to the host cell or tissue, may be introduced into a host cell or tissue, may be introduced into the host cell or tissue by causing or allowing the expression of an expression construct or vector or may be introduced into the host cell or tissue by stimulating or activating expression from an endogenous gene in the cell.

In such a cell-based method, the activity of DDAH I in the presence or absence of the test agent may be determined by assessing the ability of the DDAH I in the cell or tissue to hydrolyse a methylarginine such as ADMA. For example, the cell or tissue may be provided with ADMA or the cell or tissue may comprise ADMA. In such a cell-based method, the amount of DDAH I may be assessed in the presence or absence of a test agent in order to determine whether the agent is altering the amount of DDAH I in the cell or tissue, such as through regulation of DDAH I expression in the cell or tissue or through stabilisation of DDAH I protein within the cell or tissue. In either case, the presence of a higher DDAH I activity or an increased amount of DDAH I within the cell or tissue in the presence of the test agent indicates that the test agent may be a suitable agonist of DDAH I for use in accordance with the present invention to lower portal pressure.

In one embodiment, such a cell based assay may be carried out in vitro or ex vivo on cells or tissue deriving from the patient to be treated. It may therefore be determined whether or not the test agent is capable of increasing or maintaining the activity or amount of DDAH I in the cells or tissue of that subject. For example, such a method may be carried out on a sample of cells or tissue from the liver of the patient.

A method of the invention may use a cell-free assay. For example, the DDAH I may be present in a cell-free environment. A suitable cell-free assay may be carried out in a cell extract. For example, the contacting steps of the methods of the invention may be carried out in extracts obtained from cells that may express, produce or otherwise contain DDAH I and/or a methylarginine such as ADMA and/or a test agent. A cell-free system comprising DDAH I may be incubated with the other components of the methods of the invention such a test agent and/or a methylarginine such as ADMA. Further, the cell-free system may be incubated under conditions such that DDAH I is capable of acting to hydrolyse a methylarginine such as ADMA in the absence of the test agent. In such a cell-free method, the activity of DDAH I in the presence or absence of the test agent may be determined by assessing the ability of the DDAH I to hydrolyse a methylarginine such as ADMA. In such a cell-free method, the amount of DDAH I may be assessed in the presence or absence of a test agent in order to determine whether the agent is altering the amount of DDAH I in the cell or tissue, such as through stabilisation of DDAH I protein. In either case, the presence of a higher DDAH I activity or an increased amount of DDAH I in the presence of the test agent indicates that the test agent may be a suitable agonist of DDAH I for use in accordance with the present invention to lower portal pressure.

The contacting step(s) of the method of the invention may comprise incubation of the various components. Such incubations may be performed at any suitable temperature, typically between 4° C. and 40° C. Incubation periods may be selected for optimum activity, but may also be optimized to facilitate rapid high-throughput screening. Following the contact and optional incubation steps, the subject methods may further include a washing step to remove unbound components, where such a washing step is generally employed when required to remove label that would give rise to a background signal during detection, such as radioactive or fluorescently labelled non-specifically bound components.

Incubation in cell or cell-free assay systems may be performed in a microtiter plate (e.g. a 96-well plate or other microwell plate). Further, incubation may be performed in an automated fashion (e.g. for high-throughput screening).

A screening method of the invention may be carried out in vivo. For example, a screening method may be carried out in an animal model. In such an in vivo model, the effects of a test agent may be assessed in the liver. Preferably, the animal is a non-human animal such as a rat. For example, a screening method may be carried out in a bile duct-ligated rat model as described in the Examples. As shown in the Examples, bile duct ligation in the rat leads to a reduction in DDAH I levels in the liver of the rat. Such a model may therefore be suitable for identifying agents capable of increasing DDAH I levels. Accordingly, the screening method of the present invention may comprise the step of administering a test agent to a bile duct ligated rat and determining whether the presence of the test agent leads to an increase in the amount or activity of DDAH I in the liver of the rat.

Such a model may be used to assess the in vivo effects of a test agent. For example, such a model may be used to assess whether the test agent is capable of increasing or maintaining the activity or amount of DDAH I in vivo. In such a method, the amount of DDAH I may be assessed, the enzymatic activity may be assessed, the level of methylarginines may be assessed (e.g. ADMA) or the amount of NOS may be assessed. An in vivo model may also be used to determine whether the test agent has any unwanted side effects. For example, a method of the invention may compare the effects of a test agent on

DDAH I with its effects on other enzymes in order to determine whether the test agent is specific.

As mentioned herein, in some embodiments it may be preferable for an agent for use in the reduction of portal pressure to have no effect on the levels of pro-inflammatory cytokines or TNFα. A screening method of the invention may comprise a step of measuring levels of TNFα or pro-inflammatory cytokines in the animal after treatment with the test agent to determine whether the agent is modulating the amount of cytokines or TNFα in the animal. A screening method of the invention may comprise a step of determining whether the test agent is capable of inhibiting TNFα, wherein the ability to increase the amount or activity of DDAH I in combination with the absence of an inhibitory effect on TNFα indicates that the compound may be suitable for use in treating portal hypertension.

In such an in vivo method, the activity of DDAH I in the presence or absence of the test agent may be determined by assessing the ability of the DDAH Ito hydrolyse a methylarginine such as ADMA, or by assessing a downstream consequence of this, such as NOS levels. In such an in vivo method, the amount of DDAH I may be assessed in the presence or absence of a test agent in order to determine whether the agent is altering the amount of DDAH I in the cell or tissue, such as through stabilisation of DDAH I protein. In either case, the presence of a higher DDAH I activity or an increased amount of DDAH I in the presence of the test agent indicates that the test agent may be a suitable agonist of DDAH I for use in accordance with the present invention to lower portal pressure.

A test agent that is an agonist of DDAH I may result in an increase in DDAH I activity or levels of at least 5%, at least 10%, at least 25%, at least 50%, at least 60%, at least 75%, at least 100% or more in the presence of the test agent compared to in the absence of the test agent. Such an increase may be seen in the sample being tested or, for example where the method is carried out in an animal model, in particular tissue from the animal such as in the liver.

Levels or amounts of DDAH I may be measured by assessing expression of the DDAH I gene. Gene expression may be assessed by looking at mRNA production or levels or at protein production or levels. Expression products such as mRNA and proteins may be identified or quantified by methods known in the art. Such methods may utilise hybridisation to specifically identify the mRNA of interest. For example such methods may involve PCR or real-time PCR approaches. Methods to identify or quantify a protein of interest may involve the use of antibodies that bind that protein. For example, such methods may involve western blotting. Regulation of DDAH I gene expression may be compared in the presence and absence of a test agent. Thus test agents can be identified that increase DDAH I gene expression or that maintain DDAH I expression at a higher level than is seen in the absence of the test agent. Such test agents may be suitable agonists of DDAH I in accordance with the invention.

Activity of DDAH I may be measured by assessing levels of a product of DDAH I enzymatic activity. For example, activity of DDAH I may be measured by assessing the amount of methylarginines such as ADMA, or of downstream products such as NOS, whose levels are regulated by DDAH I. For example, DDAH activity may be assessed by determining L-citrulline formation as described in Ogawa et al (J. Biol Chem (1989) 264: 10205-10209), or as described in the Examples, or by assessing ADMA levels directly, e.g. by HPLC or tandem mass-spectroscopy.

### Pharmaceutical Formulations

A suitable DDAH I agonist as described herein is typically formulated for administration with a pharmaceutically acceptable carrier or diluent. The agonist may be any agonist as defined herein including any agonist identified by a screening method of the invention. The agonist may thus be formulated as a medicament with a standard pharmaceutically acceptable carrier(s) and/or excipient(s) as is routine in the pharmaceutical art. The exact nature of the formulation will depend upon several factors including the desired route of administration. Typically, the agonist may be formulated for oral, intravenous, intragastric, intravascular or intraperitoneal administration.

The pharmaceutical carrier or diluent may be, for example, an isotonic solution such as physiological saline. Solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners;

wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with omithine and at least one of phenylacetate and phenylbutyrate, a pharmaceutically acceptable carrier, e.g. sterile water, olive oil, ethyl oleate, glycols, e.g. propylene glycol, and if desired, a suitable amount of lidocaine hydrochloride.

Where the agonist to be administered is a nucleic acid molecule, for example where the agonist is in the form of an expression vector, certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules.

A pharmaceutical formulation in accordance with the present invention may further comprise one or more additional therapeutic agents. For example, the formulation may comprise one or more DDAH I agonists as defined herein. The formulation may comprise one or more DDAH I agonists as described here and also one or more additional therapeutic agents. Preferably the additional therapeutic agent(s) are agents which will assist in the treatment or prophylaxis of the individual to be treated. For example, one or more agents that are effective at treating portal hypertension may be administered as part of a formulation as described herein. One or more agents that are effective at treating an underlying liver condition or symptom thereof in the patient may be administered as part of a formulation as described herein.

### Treatment

The present invention provides methods for the reduction of portal blood pressure, for example in a subject with portal hypertension. Accordingly, the invention provides a method of reducing portal blood pressure comprising administering to a subject in need thereof an agonist of DDAH I. Similarly, an agonist of DDAH I may be provided for use in a method of reducing portal blood pressure. Also provided is the use of an agonist of DDAH I in the manufacture of a medicament for use in the reduction of portal blood pressure. In any of these methods and uses, the DDAH I agonist may be an agent that is not a TNFα antagonist, i.e. the agent does not reduce TNFα levels and/or does not reduce TNFα activity in the subject being treated. The DDAH I agonist may have no significant effect on TNFα levels and/or TNFα activity in the subject to be treated.

The agonist may be any agonist as described herein including any agonist identified by a screening method of the invention. The agonist may be provided in a formulation as described herein. An agonist of DDAH I as described herein is thus administered to a subject in order to reduce portal pressure in the subject. An agonist of DDAH I as described herein can thus be administered to improve the condition of a subject, for example a subject suffering from portal hypertension. An agonist of DDAH I as described herein may be administered to alleviate the symptoms of a subject, for example the symptoms associated with portal hypertension. An agonist of DDAH I as described herein may be administered to combat or delay the onset of portal hypertension or any symptom associated therewith, such as varices. The invention can therefore prevent the medical consequences of portal hypertension. Use of an agonist of DDAH I as described herein may thus extend the life of a patient with liver disease.

As described herein, the agonist of DDAH I may lead to increased expression and/or increased levels of DDAH I in the liver of the subject. For example, the agonist may be an agent that promotes transcription of DDAH I in cells of the subject or the agent may be a vector capable of expressing DDAH I in the liver of the subject. For example, DDAH I expression may be increased by a gene therapy approach whereby a polynucleotide or vector comprising a DDAH I gene as described herein is administered to the individual. The polynucleotide or vector may be targeted to the liver either through targeted administration, such as administration directly into the liver, or targeted expression such as using a liver specific promoter. Administration of such a polynucleotide or vector capable of expressing DDAH I may be used to reconstitute pathophysiologically low levels of DDAH I in an individual. This may be achieved by single or multiple administrations of such a polynucleotide or vector to the individual.

As described herein, the agonist of DDAH I may lead to increased activity of DDAH I in the liver of the individual. For example, the agonist may lead to decreased levels of asymmetric dimethylarginine (ADMA) and/or increased levels of nitric oxide synthase (NOS) in the liver of the individual.

The subject is treated with an agonist of DDAH I as described herein. As described above, the agonist of DDAH I may be administered alone or in the form of a pharmaceutical formulation. The formulation may comprise one or more agonists of DDAH I and may comprise one or more additional therapeutic or prophylactic agents.

Two or more different DDAH I agonists as described herein may be used in combination to treat a subject. The two or more agonists may be administered together, in a single formulation, at the same time, in two or more separate formulations, or separately or sequentially as part of a combined administration regimen.

An agonist or formulation of the invention may be administered by any suitable route. Preferably it is administered by oral, intravenous, intragastric, intraperitoneal or intravascular routes. The agonist or formulation may be administered directly to the liver of the subject.

The agonist is administered in a therapeutically effective amount. A suitable dose of an agonist of the invention can be determined according to various parameters such as the age, weight and condition of the subject to be treated; the type and severity of the liver disease; the route of administration; and the required regimen. A suitable dose can be determined for an individual agonist. For example, for some agonists a typical dose may be in the order of from 1 mg/kg/day to 30 g/kg/day. A physician will be able to determine the required dosage of agonist and for any particular subject.

The present invention is broadly applicable to therapeutic methods and is relevant to the development of prophylactic and/or therapeutic treatments. It is to be appreciated that all references herein to treatment include curative, palliative and prophylactic treatment.

Prophylaxis or therapy includes but is not limited to eliciting an effective increase in DDAH I amount, function or activity in order to cause a reduction in portal pressure, or in order to prevent or reduce an increase in portal pressure. For example, prophylaxis or therapy may result in the reduction of portal pressure in an subject with increased portal pressure such as a subject with portal hypertension. Prophylaxis or therapy may result in the maintenance of a particular level of portal pressure in a patient where portal pressure has been increasing or in which portal pressure is expected to increase. Prophylaxis or therapy may result in an increase in portal pressure in an individual being reduced or slowed compared to the increase that would have been seen, or would have been expected, in the absence of such treatment.

Prophylaxis or therapy may have similar effects in relation to any of the symptoms or consequences of portal hypertension described herein. That is, treatment in accordance with the present invention may lead to a lessening in the severity of such symptoms or consequences, maintenance of an existing level of such symptoms or consequences or a slowing or reduction in the worsening of such symptoms or consequences.

### Patients to be Treated

The present invention relates to the reduction of portal pressure in individuals in need thereof. A subject to be treated in accordance with the present invention may therefore have portal hypertension or may be at increased risk of portal hypertension. For example, the subject may have cirrhosis. Portal hypertension may be defined as increased blood pressure in the portal vein and its tributaries. The portal vein is the large vein that brings blood from the intestine to the liver. Portal hypertension may be defined as clinically significant when the portal pressure gradient (the difference in pressure between measurements of a catheter wedged in the hepatic veins or the portal vein and the free pressure readings in the hepatic vein or inferior vena cava) of 10 mm Hg or greater.

Methods for diagnosing portal hypertension are well known in the art and in particular to clinicians and veterinarians in the field. Preferably, the subject will have been diagnosed as having portal hypertension, for example by a medical or veterinarian professional. The subject may display one or more symptoms associated with portal hypertension.

For example, portal hypertension can lead to accumulation of ascitic fluid. This can lead to the patient's abdomen swelling. The patient may also have an enlarged spleen. An ultrasound scan may be used to examine blood flow in the portal vein and nearby blood vessels and to detect fluid in the abdomen.

Collateral vessels may be visible on the skin over the abdominal wall or around the rectum. Esophageal and gastric varices bleed easily and sometimes massively. An ultrasound or computed tomography (CT) scan can be used to look for and examine collateral vessels.

Because most people with portal hypertension also have severe liver dysfunction, they may have symptoms of liver failure, such as a tendency to bleed.

Portal pressure may be measured directly. For example, a catheter may be inserted through an incision in the neck and threaded through blood vessels into the liver or spleen to directly measure pressure in the portal blood vessels.

The individual to be treated may have been diagnosed as suffering from portal hypertension, for example by any of these methods. The individual to be treated may have been diagnosed as being at risk of portal hypertension. For example, the individual may have been diagnosed with one or more symptoms that are associated with portal hypertension. For example, portal hypertension is commonly found in patients with liver cirrhosis. The individual to be treated may have liver cirrhosis. Portal hypertension can also result from advanced liver disease including alcoholic hepatitis, idiopathic non-cirrhotic portal hypertension, congenital hepatic fibrosis, partial nodular transformation, Budd-Chiari syndrome, portal vein thrombosis, right heart failure or schistosomiasis infection. The individual to be treated may have any one or more of these conditions.

The subject to be treated may be any individual which is susceptible to increased portal pressure such as portal hypertension. The subject may be male or female. Women may be more susceptible to the adverse effects of alcohol than men. Women can develop alcoholic chronic liver disease in a shorter time frame and from smaller amounts of alcohol than men.

The subject to be treated may be a human. The subject to be treated may be a non-human animal. The subject to be treated may be a farm animal for example, a cow or bull, sheep, pig, ox, goat or horse or may be a domestic animal such as a dog or cat. The subject may or may not be an animal model for liver disease. The animal may be any age, but will often be a mature adult subject.

### EXAMPLES

### Materials and Methods

### BDL Rats

These experiments utilised an established animal model of cirrhosis, the bile duct ligated (BDL) rat. BDL rats may be generated by methods known in the art. For example, male Sprague-Dawley rats (200-250 g) may be used for this procedure. Following anaesthetisation, a mid-line laparotamy may be performed, the bile duct exposed, triply ligated with 4.0 silk suture, and severed between the second and third ligature. The wound is then closed in layers with absorbable suture, and the animal allowed to recover in a quiet room before being returned to the animal storage facility.

### DDAH-Activity Assay

The following experimental conditions were used to determine DDAH activity. Liver tissue samples (100 mg frozen tissue per 300 μL lysis buffer) were homogenized in Tris-HCl buffer in the presence of a protease inhibitor. The homogenate was centrifuged in a pre-cooled (4° C.) centrifuge for 90 min at 10,000 rpm. For the DDAH activity assay 50 μL aliquots of the resulting supernatant was added to 50 μL aliquots of PBS buffer containing 1 μL ¹⁴C L-NMMA (0.02 mCi) and 2 μL 100 mM unlabelled L-NMMA and incubated for 60 min at 37°0 C. After incubation, samples were prepared for determination of [¹⁴C] citrulline content by vortex-mixing with 1 ml of 50% (w/v) Dowex (pH 7.0) and centrifugation at 10,000 rpm for 5 min; 500 μL of the supernatant was then be mixed with 5 ml of liquid-scintillation fluid and assessed for scintillation counting on a liquid scintillation analyser (Packard Biosciences, Berks, UK.). One unit of the enzyme activity is defined as the amount that catalyzes formation of 1 μM L-citrulline from ADMA per min at 37° C.

### Molecular Analysis: Western Blot

Extracts containing equal amounts of protein were denatured and separated on 4-12% NuPAGE Bis-Tris Gels and blotted on to PVDF membranes (Invitrogen, UK). The membranes were then being incubated with using different goat anti DDAH 1&2 (1:1000, respectively) and mouse anti-eNOS and iNOS (1:500&1:10,000 respectively; Transduction Laboratories/Pharmingen, San Jose, Calif.), rabbit anti-TNF-α (1:1000; abcam), rabbit anti-ADMA (1:1000; immundiagnostik) and mouse anti-CTH (1:1000; abnova) antibodies and later with respective HRP-conjugated secondary antibodies. The bands were visualized using an enhanced ECL detection kit and quantified by densitometry. Loading accuracy was evaluated via membrane rehybridization with antibodies against mouse and rabbit anti-α tubulin (1:1000; upstate and Cell Signaling Technology, respectively).

### ADMA, SDMA and Arginine Measurement:

ADMA, SDMA and arginine were measured using fragmentation specific stable isotope dilution electrospray tandem mass spectrometry. In brief, samples de-proteinized with deuterated ADMA, SDMA and arginine, were chromatographed (acetonitrile:water, 1:1, with 0.025% formic acid) on a Teicoplanin guard column 10 mm×2.1 mm ID (Chirobiotic T, ASTEC Ltd, Congleton, UK), and analysed using a SCIEX API4000 (Applied Biosystems, Warrington, UK) in positive ion multiple reaction monitoring mode.

### Example 1

### Infliximab Treatment Increases DDAH1 Levels and Reduces Portal Pressure

Three groups of rats were used in these experiments, BDL rats treated with vehicle, BDL rats treated with the anti-TNF monoclonal antibody infliximab, and sham treated rats.

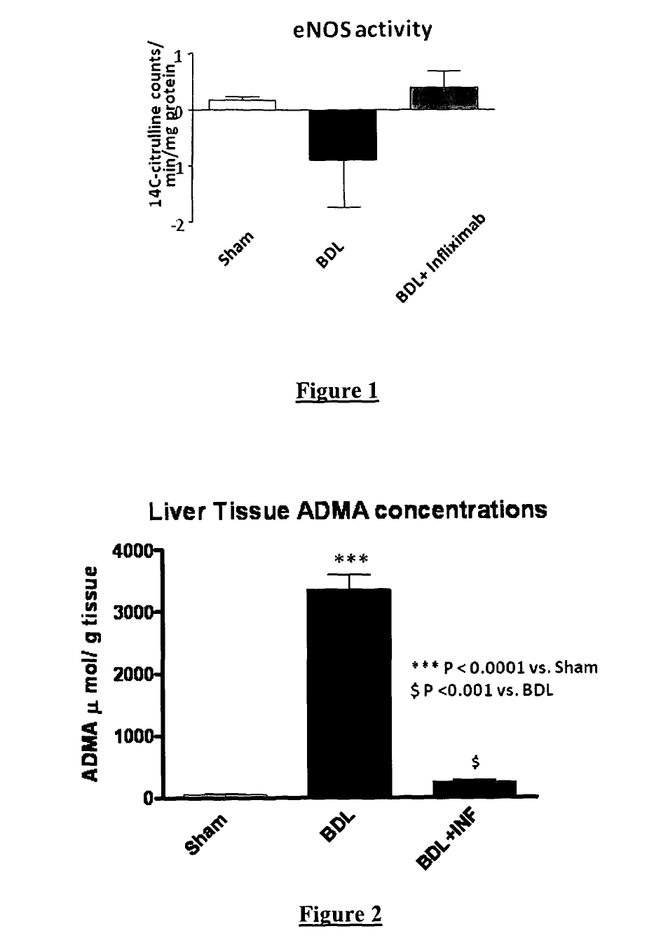

As shown in FIG. 1, bile duct ligation was found to markedly reduce eNOS activity, but treatment of BDL rats with infliximab restored eNOS activity towards similar levels to those seen in the sham animals.

As shown in FIG. 2, bile duct ligation also led to a significantly elevated liver tissue ADMA concentration compared with sham animals. However ADMA levels were substantially reduced by treatment with infliximab.

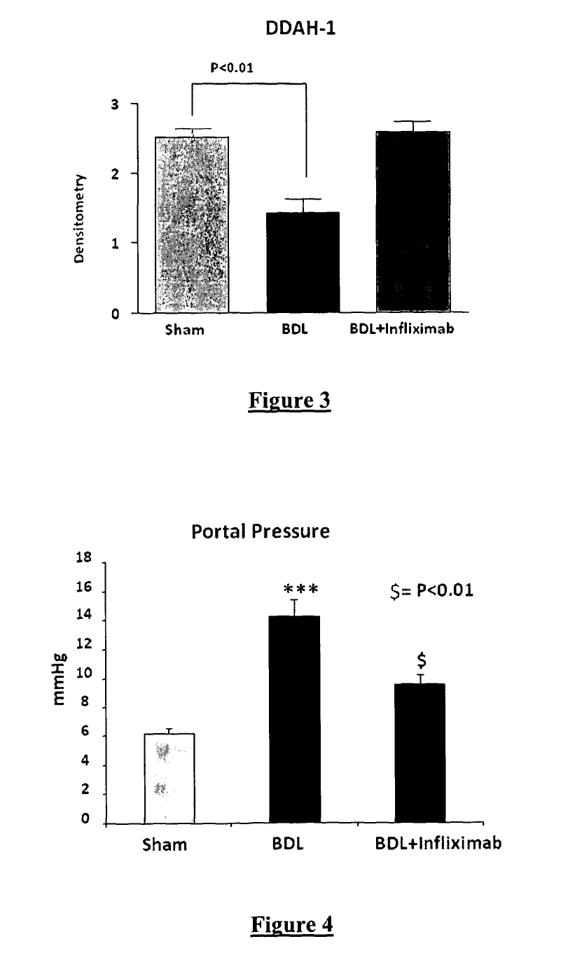

As shown in FIG. 3, bile duct ligation markedly reduced expression of the DDAH-I isoform in the livers. Upon treatment with infliximab, the DDAH-I expression levels were restored towards sham levels.

As shown in FIG. 4, portal pressure was markedly increased in BDL cirrhotic rats compared to normal sham portal pressures (14±0.7 vs. 5.5±0.3 mmHg). Following intervention with Infliximab, this was reduced by more than 30% (9.5±0.6 mmHg).

The Inventors have thus found that treatment of BDL rats with infliximab led to

A significant increase in hepatic tissue DDAH I expression.

A reduction in hepatic ADMA generation.

Increased hepatic NO generation (NOS activation).

A greater than 33% reduction in portal pressure compared to BDL rats treated with iso-volumetric saline injection.

### Example 2

### The Farnesoid Receptor Agonist INT-747 Increases DDAHI levels and Reduces Portal Pressure

The Farnesoid receptor (FXR) is a bile-acid responsive nuclear receptor previously shown to have hepatoprotective effects from bile duct ligation (BDL) injury in rats. FXR agonists have numerous target genes including DDAHI. This study tests the hypothesis that the FXR agonist (INT-747) will restore DDAH-I levels and thereby eNOS activity, lowering portal pressure.

Four weeks after BDL or sham surgery in Sprague-Dawley rats (n=14), BDL rats were gavaged with 5 mg/kg of the FXR agonist INT-747 (6-ethyl chenodeoxycholic acid, Intercept Pharmaceuticals Inc.) in vehicle (corn oil) for 5 days or with vehicle alone.

After 5 days of treatment rats underwent direct portal pressure assessment and were then sacrificed, and plasma and liver tissue collected for analysis. eNOS activity was determined radiometrically by the conversion of labelled radioactive arginine to citrulline. Protein expression for eNOS, iNOS, DDAH-1, and DDAH-2 were measured by standard Western Blotting techniques. Liver biopsies were evaluated for histopathology with H+E, Van Giesen and reticulin stains.

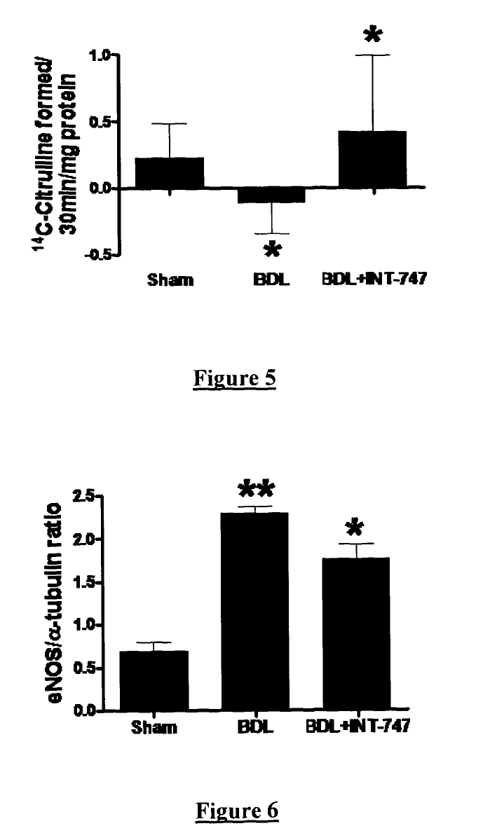

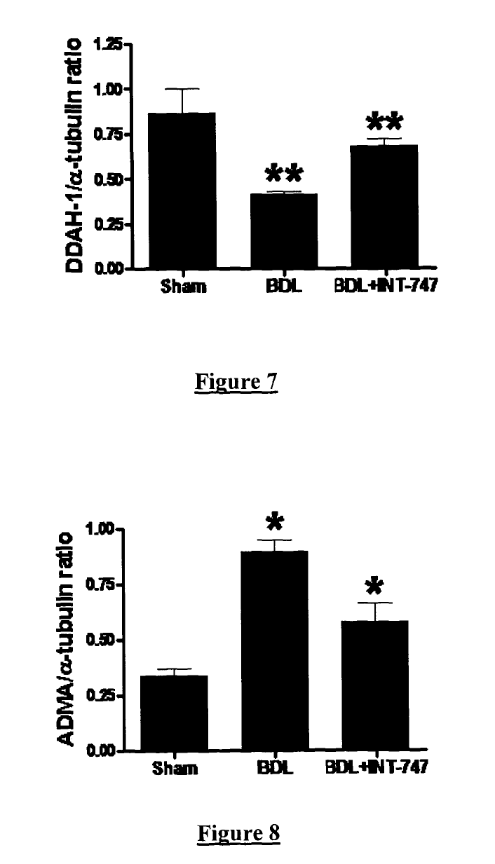

As shown in FIGS. 5 and 6, following treatment with INT-747, eNOS activity in BDL rats reverted to sham levels (*-p<0.05), with similar normalisation of eNOS protein expression (*-p<0.05)

As shown in FIGS. 7 and *, INT-747 administration to BDL rats led to a significant increase in DDAH-1 expression (**-p<0.01) with a significant reduction in ADMA (*-p<0.05) compared with BDL alone.

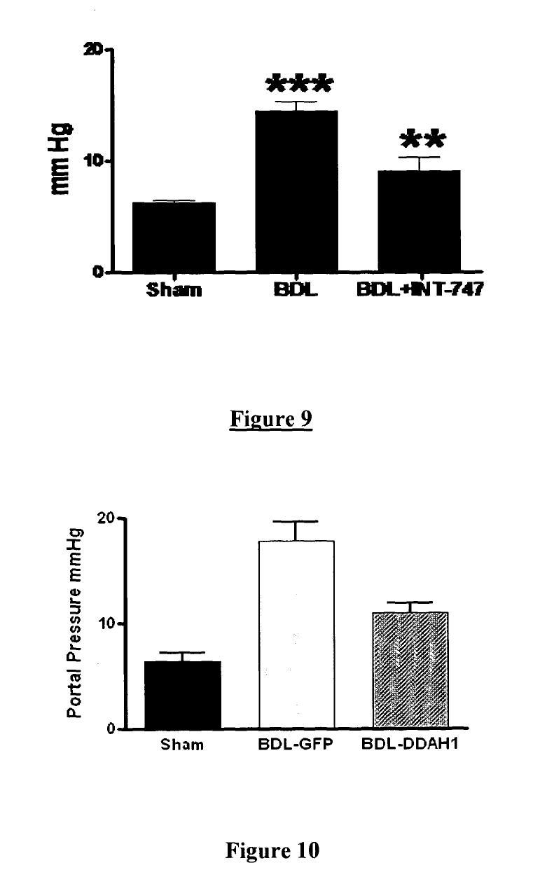

As shown in FIG. 9, INT-747 treatment in BDL rats led to a 30% reduction in portal pressure when compared to BDL+vehicle (**-p<0.01). This reduction in portal pressure following intervention with INT-747 occurred in the absence of any significant change in histological fibrosis or inflammation

Plasma TNFα levels were not significantly altered in the animals treated with INT-747 when compared with the BDL+vehicle animals.

### Example 3

The injection of naked plasmid DNA with a promoter that is efficient in mammalian cells has been demonstrated to result in effective liver transduction of the gene of interest in rodents (Maruyama et al. J Gene Med 2002, 4: 333-41). This method, termed ‘hydrodynamic gene therapy’, was used to determine the effect of transduction of DDAH-1 in BDL rats on portal pressure.

A plasmid containing human DDAH-1 cDNA was injected via a branch of the jugular vein into BDL or sham rodents produced as above. A control plasmid containing GFP was used as a control for the intervention in BDL animals. The animals underwent direct portal pressure measurement at 48 hours post intervention and plasma and tissue was collected for analysis of liver DDAH-1 mRNA (rtPCR) and protein expression (western blotting), as well as routine histology and biochemistry.

All the treated animals tolerated the hydrodynamic injection well and were given access to chow and water ad libitum. After 48 hours, at the time of sacrifice, portal pressure assessments were made. Sham rats had a mean portal pressure that was significantly lower than BDL rats injected with GFP plasmid (6.5±0.7 mmHg vs. 17.8±1.8 mmHg). However, the group that had been treated with the DDAH-1 incorporated plasmid had a 38 percent drop in portal pressure compared with the GFP treated group (11±0.9 mmHg). See FIG. 10. This data confirms the assertion that genetic therapy by reconstitution of DDAH-1 cDNA may act as a therapy for the treatment of portal hypertension.

### Example 4

Adeno-associated virus (AAV) vectors are amongst the most frequently used viral vectors for gene therapy. Efficient liver transduction of the AAV2 genome with an AAV8 capsid (AAV2/8) has been demonstrated, when injected intravenously into mice (Osman et al; Atherosclerosis 2009, 204: 121-6). Expression of the gene of interest was driven by a truncated liver-specific promoter, LPI, containing segments of the human apoE/CI hepatic control region (HCR) and alpha-1-antitrypsin (hAAT) gene promoter, providing strong hepatic-restricted transgene production.

We have cloned a DNA construct containing human DDAH1 cDNA in the AAV2/8 plasmid, under the control of the liver-specific LPI promoter.

The subsequent steps in creating the AAV vector are: (a) transfection of the DDAH-AAV2/8 plasmid and helper and packaging plasmids into competent cells, (b) purification of the DDAH-AAV2/8 with AVB Sepharose Column, and (c) quantification of DDAH-AAV2/8 with rtPCR.

The DDAH-AAV2/8 vector is injected via tail vein injection into BDL or sham rats. A negative control AAV2/8, without the gene of interest, is also injected into BDL rats.

Portal pressure may be assessed by direct cannulation. Transduction of DDAH-1 may be assessed by measurement of mRNA (rtPCR) and protein (western blotting).

### Example 5

Adenovirus vectors have been shown to demonstrate efficient transduction of hepatocytes and other cell types in rodent models of liver disease (Yu et al, Am J Phys 2002, 282: G565-G572; Garcia-Banuelos et al Gene Therapy 2002, 9: 127-134).

An adenovirus expressing DDAH-1 is constructed. Once constructed the DDAH-adenovirus construct may be used to determine the effect of transduction of DDAH-1 into hepatocytes and non-parenchymal cells (including sinusoidal endothelial cells) on eNOS activity portal pressure in BDL rats.

The DDAH-adenovirus vector is injected via tail vein injection into BDL sham rats. A negative control AAV2/8, without the gene of interest, is also injected into BDL rats. Portal pressure may be assessed at 5 days by direct cannulation of the portal vein. Transduction of DDAH-1 may be assessed by measurement of mRNA (rtPCR) and protein (western blotting).

1. A method of reducing portal blood pressure comprising administering to a subject in need thereof an agonist of DDAH I, wherein said agonist does not reduce plasma TNFα levels in the subject.

2. A method according to claim 1 wherein said agonist leads to:
a) increased expression of DDAH I in the liver of the subject; and/or
b) increased levels of DDAH I in the liver of the subject.

3. A method according to claim 2 wherein said agonist promotes transcription of DDAH I in cells of the subject.

4. A method according to claim 2 wherein said agonist is a vector capable of expressing DDAH I in the liver of the subject.

5. A method according to claim 1 wherein said agonist increases the activity of DDAH I in the liver of the individual.

6. A method according to claim 5 wherein said agonist leads to:
a) decreased levels of asymmetric dimethylarginine (ADMA); and /or
b) increased levels of nitric oxide synthase (NOS);
in the liver of the individual.

7. A method according to claim 1 wherein said subject has portal hypertension.

8. A method according to claim 1 wherein said subject has liver cirrhosis.

9. A method of identifying an agent suitable for use in treating portal hypertension, the method comprising determining whether a test agent is capable of increasing or maintaining the amount or activity of DDAH I, wherein the ability to increase the amount or activity of DDAH I indicates that the compound may be suitable for use in treating portal hypertension.

10. A method according to claim 9, comprising a step of contacting a cell or tissue comprising DDAH I with a test agent and determining whether the presence of the test agent leads to an increase in the amount or activity of DDAH I in the cell or tissue.

11. A method according to claim 9 wherein the amount or activity of DDAH I is assessed in the liver or in tissue or cells derived from the liver.

12. A method according to claim 9, comprising the step of administering a test agent to a bile duct ligated rat and determining whether the presence of the test agent leads to an increase in the amount or activity of DDAH I in the liver of the rat.

13. A method according to claim 9, further comprising determining whether said test agent is capable of inhibiting TNFα, wherein the ability to increase the amount or activity of DDAH I in combination with the absence of an inhibitory effect on TNFα indicates that the compound may be suitable for use in treating portal hypertension.
